(19) United States
(12) Patent Application Publication
(10) Pub. No.: US 2026/0155220 A1
(43) Pub. Date: Jun. 4, 2026

(54) SYSTEM FOR DETERMINING A MEDICAL IMAGE CORRESPONDING TO A RADIOLOGY REPORT USING ARTIFICIAL INTELLIGENCE MODELS

(71) Applicant: GE Precision Healthcare LLC, Waukesha, WI (US)

(72) Inventors: Sarthak PANDEY, Bengaluru (IN); Deepa ANAND, Bengaluru (IN); Dattesh SHANBHAG, Bengaluru (IN); Rakesh MULLICK, Bengaluru (IN)

(21) Appl. No.: 18/967,196

(22) Filed: Dec. 3, 2024

(51) Int. Cl. G16H 15/00

(52) U.S. Cl. CPC G16H 15/00

(57) ABSTRACT

A system may receive a radiology report including a finding of a radiologist with respect to a region of interest of a subject, and generate, using a first AI model, structured data including a set of predetermined fields and a set of corresponding values extracted from the radiology report. The system may determine, using the first AI model, a medical image dataset of the subject corresponding to the radiology report stored in a medical image database using the structured data. The system may segment and label, using a second AI model, a set of regions of each medical image of the medical image dataset. The system may determine, using the first AI model, a medical image of the medical image dataset that depicts the region of interest of the subject using the structured data, and perform an action based on determining the medical image that depicts the region of interest.

### TECHNICAL FIELD

The present disclosure relates, generally, to a system for determining a medical image corresponding to a radiology report using artificial intelligence (AI) models. More specifically, the present disclosure relates to a system that receives a radiology report of a subject related to a medical imaging test of the subject, determines a medical image dataset of the subject stored in a medical image database corresponding to the medical imaging test, and determines a particular medical image that corresponds to the medical imaging test. The system may perform an action based on the determined medical image, such as displaying the radiology report and the determined medical image via a user interface for a radiologist to view, generating a data entry that correlates the radiology report and the determined medical image and storing the data entry in a database, or the like.

### BACKGROUND

For a medical imaging test, a medical imaging system may acquire medical images of a subject to permit assessment or diagnosis of the subject. A radiologist may review the medical images, and generate a radiology report for the medical imaging test. The radiology report may include various information, such as a medical history of the subject, a date of the medical imaging test, an imaging modality of the medical imaging device that acquired the medical images of the medical imaging test, findings of the radiologist, comparison of the findings with previous findings for the subject, recommendations of the radiologist, etc. In some cases, a subject may be associated with a large number of medical imaging tests and a corresponding large number of medical image datasets and radiology reports. The medical imaging tests might have been performed using medical imaging devices having different imaging modalities, might have been performed using different imaging protocols, might have been performed in association with different regions that were imaged, or the like. A radiologist that intends to review a particular radiology report might find it difficult to determine a particular medical image dataset that corresponds to the radiology report, and might also find it difficult to determine a particular medical image that depicts a region of interest corresponding to the radiology report. In this case, the radiologist might need to review an excessive number of medical image datasets and corresponding underlying medical images to determine an appropriate medical image that corresponds to the findings in the radiology report, which might increase the time associated with the radiologist's review, might decrease the efficiency of the radiologist, etc. Further, the review of a large number of medical image datasets might consume device, network, and database resources. Accordingly, there is a need for a technique that improves the retrieval of one or more medical images corresponding to a radiology report.

### SUMMARY

This summary introduces concepts that are described in more detail in the detailed description. It should not be used to identify essential features of the claimed subject matter, nor to limit the scope of the claimed subject matter.

In an aspect, a system may include a memory configured to store instructions; and one or more processors configured to execute the instructions to: receive a radiology report including a finding of a radiologist with respect to a region of interest of a subject; generate, using a first artificial intelligence (AI) model, structured data including a set of predetermined fields and a set of corresponding values extracted from the radiology report; determine, using the first AI model, a medical image dataset of the subject corresponding to the radiology report from among a plurality of medical image datasets of the subject stored in a medical image database using the structured data; segment, using a second AI model, a set of regions of each medical image of the medical image dataset; label, using the second AI model, the set of regions of each medical image of the medical image dataset; determine, using the first AI model, a medical image of the medical image dataset that depicts the region of interest of the subject using the structured data and the labelled and segmented set of regions; and perform an action based on determining the medical image that depicts the region of interest of the subject.

In another aspect, a method may include receiving a radiology report including a finding of a radiologist with respect to a region of interest of a subject; generating, using a first artificial intelligence (AI) model, structured data including a set of predetermined fields and a set of corresponding values extracted from the radiology report; determining, using the first AI model, a medical image dataset of the subject corresponding to the radiology report from among a plurality of medical image datasets of the subject stored in a medical image database using the structured data; segmenting, using a second AI model, a set of regions of each medical image of the medical image dataset; labelling, using the second AI model, the set of regions of each medical image of the medical image dataset; determining, using the first AI model, a medical image of the medical image dataset that depicts the region of interest of the subject using the structured data and the labelled and segmented set of regions; and performing an action based on determining the medical image that depicts the region of interest of the subject.

In yet another aspect, a non-transitory computer-readable medium may store instructions that, when executed by one or more processors, cause the one or more processors to: receive a radiology report including a finding of a radiologist with respect to a region of interest of a subject; generate, using a first artificial intelligence (AI) model, structured data including a set of predetermined fields and a set of corresponding values extracted from the radiology report; determine, using the first AI model, a medical image dataset of the subject corresponding to the radiology report from among a plurality of medical image datasets of the subject stored in a medical image database using the structured data; segment, using a second AI model, a set of regions of each medical image of the medical image dataset; label, using the second AI model, the set of regions of each medical image of the medical image dataset; determine, using the first AI model, a medical image of the medical image dataset that depicts the region of interest of the subject using the structured data and the labelled and segmented set of regions; and perform an action based on determining the medical image that depicts the region of interest of the subject.

### BRIEF DESCRIPTION OF DRAWINGS

FIG. 1 is a diagram of an example system 100 for determining a medical image corresponding to a radiology report using AI models.

FIG. 2 is a diagram of example components of one or more devices of FIG. 1.

FIG. 3 is a diagram of an example platform of FIG. 1 and example AI models of the platform of FIG. 1.

FIG. 4 is a diagram is a flowchart of an example process for determining a medical image corresponding to a radiology report using AI models.

FIGS. 5A-5E are diagrams of an example process for determining a medical image corresponding to a radiology report using AI models.

FIG. 6 is a diagram of an example process for generating structured data including a set of predetermined fields and a set of corresponding values extracted from a radiology report.

FIGS. 7A-7E are diagrams of an example process for determining a medical image corresponding to a radiology report using AI models.

### DETAILED DESCRIPTION

As described above, a radiologist that intends to review a particular radiology report might find it difficult to determine a particular medical image dataset that corresponds to the radiology report, and might also find it difficult to determine a particular medical image that depicts a region of interest corresponding to the radiology report. For instance, the radiologist might find it difficult to determine a particular medical image that corresponds to a finding in the radiology report. In this case, the radiologist might need to review an excessive number of medical image datasets and corresponding underlying medical images to determine an appropriate medical image that corresponds to the findings in the radiology report, which might increase the time associated with the radiologist's review, might decrease the efficiency of the radiologist, etc. Further, the review of the radiologist may consume device, network, and database resources.

Some embodiments herein provide a system for determining a medical image corresponding to a radiology report using AI models. For instance, some embodiments herein provide a system that receives a radiology report including a finding of a radiologist with respect to a region of interest of a subject, and generates, using a first AI model (e.g., a large language model (LLM)), structured data including a set of predetermined fields and a set of corresponding values extracted from the radiology report. Further, some embodiments herein provide a system that determines, using the first AI model, a medical image dataset of the subject corresponding to the radiology report from among a plurality of medical image datasets of the subject stored in a medical image database using the structured data. Further still, some embodiments herein provide a system that segments, using a second AI model (e.g., a foundation model), a set of regions of each medical image of the medical image dataset, and labels, using the second AI model, the set of regions of each medical image of the medical image dataset. Further still, some embodiments herein provide a system that determines, using the first AI model, a medical image of the medical image dataset that depicts the region of interest of the subject using the structured data and the labelled and segmented set of regions, and performs an action based on determining the medical image that depicts the region of interest of the subject. In this way, some embodiments herein provide an improvement in the technical field of medical image retrieval by determining a particular medical image that corresponds to the findings in a radiology report using AI models, which, among other things, reduces the amount of requests for medical image datasets to a database. Further, in this way, some embodiments herein provide an improvement to user devices, databases, and network associated with medical image retrieval by permitting a particular medical image that corresponds to the findings in a radiology report to be determined using AI models, which, among other things, reduces the amount of requests for medical image datasets to a database.

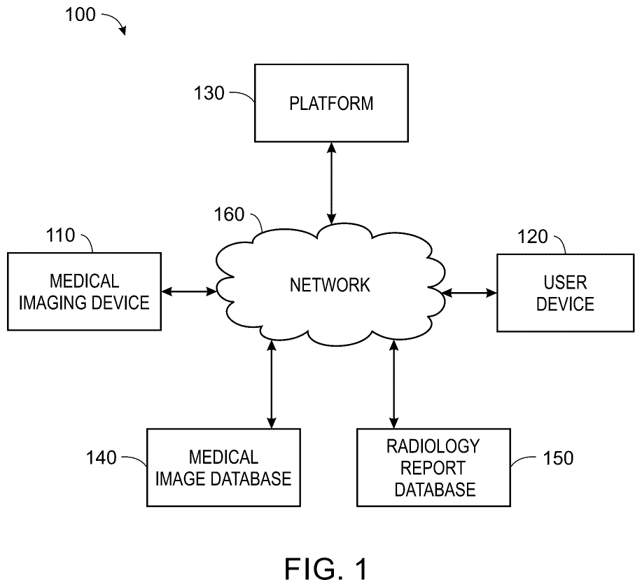

FIG. 1 is a diagram of an example system 100 for determining a medical image corresponding to a radiology report using AI models. As shown in FIG. 1, the system 100 may include a medical imaging device 110, a user device 120, a platform 130, a medical image database 140, a radiology report database 150, and a network 160.

The medical imaging device 110 may be configured to acquire a medical image of a region of interest of a subject using a particular imaging modality for a medical imaging test. For example, the medical imaging device 110 may be a computed tomography (CT) device that is configured to acquire CT images, a magnetic resonance imaging (MRI) device that is configured to acquire MRI images, an ultrasound device that is configured to acquire ultrasound images, an X-ray device that is configured to acquire X-ray images, a positron emission tomography (PET) device that is configured to acquire PET images, or the like. The subject may be a patient, an animal, a phantom, or the like. The region of interest may be any anatomical region of the subject. For example, the region of interest may be the brain, the heart, the liver, the pancreas, the kidneys, etc.

The user device 120 may be configured to display a medical image of a region of interest of a subject for viewing by a radiologist, display a radiology report for viewing by a radiologist, or the like. For example, the user device 120 may be a desktop computer, a laptop computer, a medical device, a tablet computer, a smartphone, or the like.

The platform 130 may be configured to receive a radiology report including a finding of a radiologist with respect to a region of interest of a subject; generate, using a first AI model, structured data including a set of predetermined fields and a set of corresponding values extracted from the radiology report; determine, using the first AI model, a medical image dataset of the subject corresponding to the radiology report from among a plurality of medical image datasets of the subject stored in a medical image database using the structured data; segment, using a second AI model, a set of regions of each medical image of the medical image dataset; label, using the second AI model, the set of regions of each medical image of the medical image dataset; determine, using the first AI model, a medical image of the medical image dataset that depicts the region of interest of the subject using the structured data and the labelled and segmented set of regions; and perform an action based on determining the medical image that depicts the region of interest of the subject. For example, the platform 130 may be a server, a computer, or the like.

The medical image database 140 may be configured to store medical image datasets acquired by the medical imaging devices 110. For example, the medical image database 140 may be a cloud database, a hierarchical database, a network database, a centralized database, a picture archiving and communication system (PACS), or the like.

The radiology report database 150 may be configured to store radiology reports generated by the platform 130, store radiology reports generated by radiologists, or the like. For example, the radiology report database 150 may be a cloud database, a hierarchical database, a network database, a centralized database, or the like.

The network 160 may permit communication between the medical imaging device 110, the user device 120, the platform 130, the medical image database 140, and/or the radiology report database 150. For example, the network 160 may be a local area network (LAN), a wide area network (WAN), a metropolitan area network (MAN), a cellular network, a private network, an ad hoc network, an intranet, the Internet, a fiber optic-based network, a wired network, a wireless network, or the like, and/or a combination of these or other types of networks.

The number and arrangement of the system 100 are provided as an example. In practice, the system 100 may include additional devices, fewer devices, different devices, or differently arranged devices than those shown in FIG. 1. Additionally, or alternatively, a set of devices (e.g., one or more devices) of the system 100 may be integrated into a single devices, and/or perform one or more functions described as being performed by another devices, or set of devices, of the system 100.

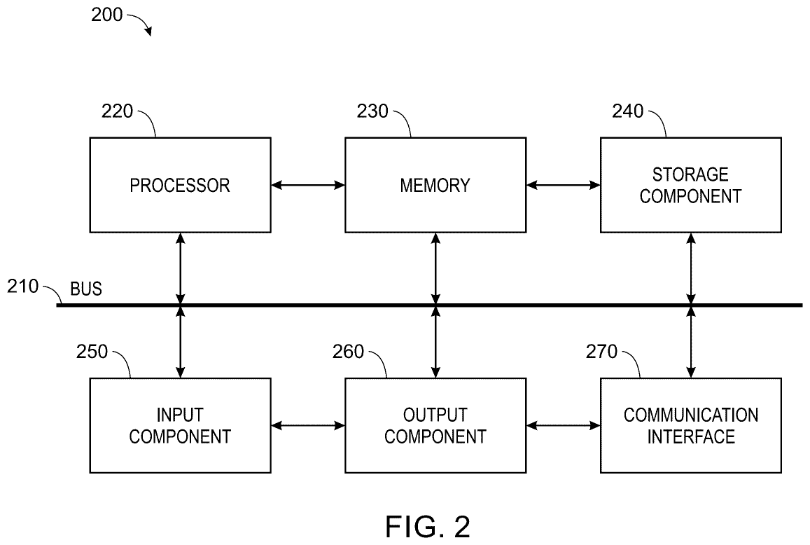

FIG. 2 is a diagram of example components of one or more devices 200 of FIG. 1. The device 200 may correspond to the medical imaging device 110, the user device 120, the platform 130, the medical image database 140, and/or the radiology report database 150. As shown in FIG. 2, the device 200 may include a bus 210, a processor 220, a memory 230, a storage component 240, an input component 250, an output component 260, and a communication interface 270.

The bus 210 includes a component that permits communication among the components of the device 200. The processor 220 may be implemented in hardware, firmware, or a combination of hardware and software. The processor 220 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 220 may include one or more processors capable of being programmed to perform a function. The processor 220 may include one or more processors 220 configured to perform the operations described herein. For example, a single processor 220 may be configured to perform all of the operations described herein. Alternatively, multiple processors 220, collectively, may be configured to perform all of the operations described herein, and each of the multiple processors 220 may be configured to perform a subset of the operations described herein. For example, a first processor 220 may perform a first subset of the operations described herein, a second processor 220 may be configured to perform a second subset of the operations described herein, etc.

The memory 230 may include a random access memory (RAM), a read only memory (ROM), and/or another type of dynamic or static storage device (e.g., a flash memory, a magnetic memory, and/or an optical memory) that stores information and/or instructions for use by the processor 220.

The storage component 240 may store information and/or software related to the operation and use of the device 200. For example, the storage component 240 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 250 may include a component that permits the device 200 to receive information, such as via user input (e.g., a touch screen display, a keyboard, a keypad, a mouse, a button, a switch, a camera, and/or a microphone). Additionally, or alternatively, the input component 250 may include a sensor for sensing information (e.g., a global positioning system (GPS) component, an accelerometer, a gyroscope, and/or an actuator). The output component 260 may include a component that provides output information from the device 200 (e.g., a display, a speaker for outputting sound at the output sound level, and/or one or more light-emitting diodes (LEDs)).

The communication interface 270 may include a transceiver-like component (e.g., a transceiver and/or a separate receiver and transmitter) that enables the device 200 to communicate with other systems, such as via a wired connection, a wireless connection, or a combination of wired and wireless connections. The communication interface 270 may permit the device 200 to receive information from another system and/or provide information to another system. For example, the communication interface 270 may include an Ethernet interface, an optical interface, a coaxial interface, an infrared interface, a radio frequency (RF) interface, a universal serial bus (USB) interface, a Wi-Fi interface, a cellular network interface, or the like.

The device 200 may perform one or more processes described herein. The device 200 may perform these processes based on the processor 220 executing software instructions stored by a non-transitory computer-readable medium, such as the memory 230 and/or the storage component 240. A computer-readable medium may be defined herein as a non-transitory memory device. A memory device may include memory space within a single physical storage device or memory space spread across multiple physical storage devices.

The software instructions may be read into the memory 230 and/or the storage component 240 from another computer-readable medium or from another system via the communication interface 270. When executed, the software instructions stored in the memory 230 and/or the storage component 240 may cause the processor 220 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of the components of the device 200 shown in FIG. 2 are provided as an example. In practice, the device 200 may include additional components, fewer components, different components, or differently arranged components than those shown in FIG. 2. Additionally, or alternatively, a set of components (e.g., one or more components) of the device 200 may perform one or more functions described as being performed by another set of components of the device 200.

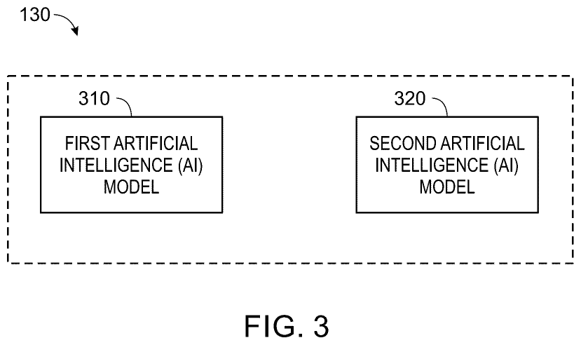

FIG. 3 is a diagram of an example platform 130 of FIG. 1, and example AI models of the platform of FIG. 1. As shown in FIG. 3, the platform 130 may include a first AI model 310 and a second AI model 320.

According to an embodiment, the first AI model 310 may be configured to receive a radiology report including a finding of a radiologist with respect to a region of interest of a subject as an input, and generate structured data including a set of predetermined fields and a set of corresponding values extracted from the radiology report as an output. Additionally, or alternatively, the first AI model 310 may be configured to determine a medical image dataset of a subject corresponding to a radiology report from among a plurality of medical image datasets of the subject stored in the medical image database 140 using the structured data. Additionally, or alternatively, the first AI model 310 may be configured to determine a medical image of the medical image dataset that depicts the region of interest of the subject using the structured data and a labelled and segmented set of regions. For example, the first AI model 310 may be an LLM, a generative AI model, a generative pre-trained transformer model, a bidirectional encoder representations from transformers model, an embeddings model, a text recognition model, a text classification model, or the like.

According to an embodiment, the second AI model 320 may be configured to segment a set of regions of each medical image of the medical image dataset. Additionally, or alternatively, the second AI model 320 may be configured to label the set of regions of each medical image of the medical image dataset. For example, the second AI model 320 may be a foundation model, convolutional neural network (CNN) model, an edge-based segmentation model, a clustering-based segmentation model, a neural network-based segmentation model, a region-based segmentation model, a residual neural network, a random forest model, a decision tree model, an artificial neural network (ANN), a Naïve Bayes model, a decision tree, a recurrent neural network (RNN), a logistic regression model, a support vector machine, or the like.

According to an embodiment, the first AI model 310 and/or the second AI model 320 may be associated with a training phase, a deployment phase, and a monitoring phase. In the training phase, the platform 130 may receive and process training data to generate a trained model (which may be the first AI model 310 or the second AI model 320). The training data may be generated, received, or otherwise obtained from internal and/or external resources.

Generally, the trained model may include a set of variables (e.g., nodes, neurons, filters, or the like) that are tuned (e.g., weighted, biased, or the like) to different values via the application of the training data. According to an embodiment, the training process may employ supervised, unsupervised, semi-supervised, and/or reinforcement learning processes to train the model. According to an embodiment, a portion of the training data may be withheld during training and/or used to validate the trained model.

For supervised learning processes, the training data may include labels or scores that may facilitate the training process by providing a ground truth. For example, the labels or scores may indicate an output of the model. Training may proceed by feeding a training dataset including the training data into the model. The model may have variables set at initialized values (e.g., at random, based on Gaussian noise, based on pre-trained values, or the like). The model may generate an output based on the training dataset being input to the model. The output may be compared with the corresponding label or score (e.g., the ground truth) indicating the known output, which may then be back-propagated through the model to adjust the values of the variables. This process may be repeated for a plurality of samples at least until a determined loss or error is below a predefined threshold. According to an embodiment, some of the training data may be withheld and used to further validate or test the trained model.

For unsupervised learning processes, the training data may not include pre-assigned labels or scores to aid the learning process. Instead, unsupervised learning processes may include clustering, classification, or the like, to identify naturally occurring patterns in the training data. As an example, the training data may be clustered into groups based on identified similarities and/or patterns. K-means clustering or K-Nearest Neighbors may also be used, which may be supervised or unsupervised. Combinations of K-Nearest Neighbors and an unsupervised cluster technique may also be used. For semi-supervised learning, a combination of training data with pre-assigned labels or scores and training data without pre-assigned labels or scores may be used to train the model.

When reinforcement learning is employed, an agent (e.g., an algorithm) may be trained to make a decision from the training data through trial and error. For example, based on making a decision, the agent may then receive feedback (e.g., a positive reward if the prediction was above a predetermined threshold), adjust its next decision to maximize the reward, and repeat until a loss function is optimized.

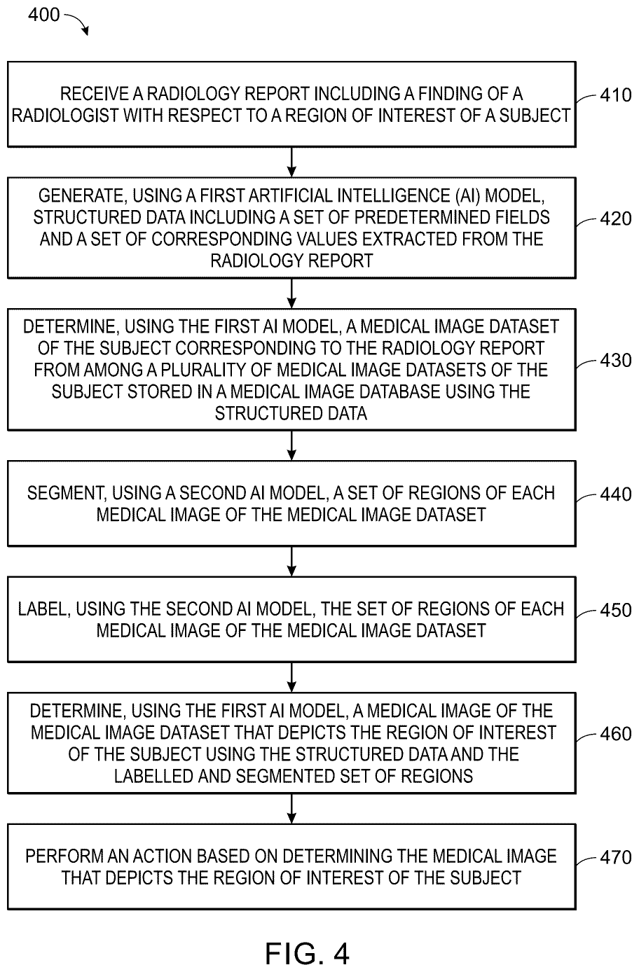

After being trained, the trained model may be stored and subsequently applied by the platform 130 during the deployment phase. For example, during the deployment phase, the trained model executed by the platform 130 may receive input data. During the deployment phase, the trained model may perform one or more operations as described in connection with FIG. 4.

After being deployed, the trained model may be monitored during the monitoring phase. For example, during the monitoring phase, the model may generate monitoring data that is used to monitor the trained model. The monitoring data may include data that identifies an output as determined by an operator. During the monitoring phase, monitoring data may be analyzed along with the predicted output data and input data to determine an accuracy of the trained model. According to an embodiment, based on the analysis, the process may return to the training phase, where values of one or more variables of the model may be adjusted to improve the accuracy of the model.

The number and arrangement of the AI models shown in FIG. 3 are provided as an example. In practice, the platform 130 may include additional AI models, fewer AI models, different AI models, or differently arranged AI models than those shown in FIG. 3. Additionally, or alternatively, a set of AI models (e.g., one or more AI models) of the platform 130 may perform one or more functions described as being performed by another set of AI models of the platform 130.

FIG. 4 is a diagram is a flowchart of an example process for determining a medical image corresponding to a radiology report using AI models. According to an embodiment, the platform 130 may be configured to perform one or more operations of the process 400. Alternatively, one or more other devices of FIG. 1 may be configured to perform one or more operations of the process 400.

As shown in FIG. 4, the process 400 may include receiving a radiology report including a finding of a radiologist with respect to a region of interest of a subject (operation 410). For example, the platform 130 may receive a radiology report from the user device 120, from the radiology report database 150, or the like. The platform 130 may receive the radiology report based on an input from the user device 120 that selects a particular radiology report for viewing, based on the radiology report being generated, based on the radiology report being stored in the radiology report database 150, based on a predetermined timeframe, or the like. The radiology report may include one or more sections. For example, the radiology report may include an imaging modality section that identifies the imaging modality for the medical imaging test, a date section that identifies a date of the medical imaging test, a time section that identifies a time of the medical imaging test, or the like. Additionally, or alternatively, the radiology report may include a reason section that identifies a reason for the medical imaging test, such as symptoms, previous medical history, or the like. Additionally, or alternatively, the radiology report may include a comparison section that includes information from a previous medical imaging test. Additionally, or alternatively, the radiology report may include a protocol section that identifies a protocol of the medical imaging test. Additionally, or alternatively, the radiology report may include a findings section that identifies a finding of the radiologist, such as what the radiologist sees in the medical image. For instance, the findings section may include a structure of a region of interest, and an associated description of the structure. As an example, the findings section may include a description of an area (e.g., “liver”), and an associated description of the structure (e.g., “normal”). The findings section may include a region section that identifies a region to which the finding is pertinent. Further, the findings section may include an anatomy section that identifies the anatomy to which the finding is pertinent. Further still, the findings section may include a laterality section that identifies a laterality of the finding. Additionally, or alternatively, the radiology report may include an impression section that identifies a summary of the findings, potential causes, differential diagnoses, recommendations, or the like.

As further shown in FIG. 4, the process 400 may include generating, using a first artificial intelligence (AI) model, structured data including a set of predetermined fields and a set of corresponding values extracted from the radiology report (operation 420). For example, the platform 130 may generate, using the first AI model 310, structured data including a set of predetermined set of fields and a set of corresponding values extracted from the radiology report. According to an embodiment, the structured data may include a predetermined format. For example, the predetermined format may be JavaScript Object Notation (JSON), Extensible Markup Language (XML), or the like. The predetermined set of fields may include a date field that identifies a date of the medical imaging test, an imaging modality field that identifies an imaging modality of the medical imaging device 110 that acquired the medical images, a protocol field that identifies a protocol of the medical imaging test, a findings field that identifies a finding of the medical imaging test, a region field that identifies a region of interest to which the finding pertains, an anatomy field that identifies the anatomical feature to which the finding pertains, or the like. According to an embodiment, the platform 130 may be preconfigured with the set of predetermined fields. Additionally, or alternatively, the platform 130 may receive a configuration instruction that identifies the predetermined set of fields. The platform 130 may input the radiology report into the first AI model 130, and receive the structured data including a set of predetermined fields and a set of corresponding values extracted from the radiology report based on an output of the first AI model 310. The first AI model 310 may be configured to generate the structured data including the set of predetermined fields and the set of corresponding values extracted from the radiology report. The set of corresponding values may be particular values extracted from the radiology report that correspond to the set of predetermined fields.

As further shown in FIG. 4, the process 400 may include determining, using the first AI model, a medical image dataset of the subject corresponding to the radiology report from among a plurality of medical image datasets of the subject stored in a medical image database using the structured data (operation 430). For example, the platform 130 may determine, using the first AI model 310, a medical image dataset of the subject corresponding to the radiology report from among a plurality of medical image datasets of the subject stored in the medical image database 140. The platform 130 may input the structured data and information associated with the plurality of medical image datasets into the first AI model 130, and determine the medical image dataset based on an output of the first AI model 130. The information associated with the plurality of medical image datasets may be metadata of the medical image datasets, header information of the medical image datasets, or the like. The first AI model 130 may be configured to determine the medical image dataset using one or more fields of the structured data. For example, the first AI model 130 may determine a medical image dataset that includes a same date as included in the date field of the structured data, that includes a same imaging modality as included in the imaging modality field of the structured data, that includes a same region as included in a region field of the structured data, or the like. According to an embodiment, the first AI model 130 may compare the structured data and the information associated with the plurality of medical image datasets, and select a particular medical image dataset that most closely matches the structured data. Additionally, or alternatively, the platform 130 may generate a prompt for the first AI model 130, and input the prompt to the first AI model 130 which causes the first AI model 130 to select the particular medical image dataset. For example, the prompt may identify the potential medical image datasets, and may identify values of the structured data.

As further shown in FIG. 4, the process 400 may include segmenting, using a second AI model, a set of regions of each medical image of the medical image dataset (operation 440). For example, the platform 130 may segment, using the second AI model 320, a set of regions of each medical image of the medical image dataset. The platform 130 may input each medical image of the medical image dataset into the second AI model 320, and receive segmented medical images based on an output of the second AI model 320. The segmented medical images may include one or more segmented structures. The segmented structures may be tissues, vessels, tumors, lesions, anomalies, or the like. Additionally, or alternatively, the platform 130 may input each segmented medical image of the medical image dataset into the second AI model 320, and receive classified medical images based on an output of the second AI model 320. The classified medical images may include one or more classified structures. A classified structure may include a classification, such as benign, malignant, anomalous, or the like.

As further shown in FIG. 4, the process 400 may include labelling, using the second AI model, the set of regions of each medical image of the medical image dataset (operation 450). For example, the platform 130 may label the set of regions of each medical image of the medical image dataset. The platform 130 may input the segmented medical images and/or the classified medical images into the second AI model 320, and receive labelled medical images based on an output of the second AI model 320. A label may identify a structure depicted in a medical image, a classification of the structure, a location of the structure, a viewpoint of the structure, or the like.

As further shown in FIG. 4, the process 400 may include determining, using the first AI model, a medical image of the medical image dataset that depicts the region of interest of the subject using the structured data and the labelled and segmented set of regions (operation 460). For example, the platform 130 may determine, using the first AI model 310, a medical image of the medical image dataset that depicts the region of interest of the subject using the structured data and the labelled and segmented set of regions. The platform 130 may input the structured data and the labelled and segmented set of regions into the first AI model 310, and receive information identifying a medical image of the medical image dataset that depicts the region of interest of the subject based on an output of the first AI model 310. The first AI model 310 may compare one or more values of one or more corresponding fields of the structured data with the labelled and segmented set of regions, and determine a medical image of the medical image dataset that depicts the region of interest. According to an embodiment, the platform 130 may determine a medical image that includes a label that exactly matches a value of a field of the structured data. For example, if the structured data includes a value of “mitral valve” for the region of interest and a medical image includes a label of “mitral valve” for a segmented structure, then the platform 130 may determine the medical image as depicting the region of interest. According to another embodiment, the platform 130 may determine a medical image that includes a label that generally matches a value of a field of the structured data. For example, if the structured data includes a value of “mitral valve” for the region of interest and a medical image includes a label of “atrioventricular valve” for a segmented structure, then the platform 130 may determine the medical image as depicting the region of interest. According to an embodiment, the platform 130 may select a medical image that includes a greatest number of matches between labels and values of the structured data. For example, the platform 130 may determine a score of a medical image based on a number of matches between labels and values of the structured data, and select the medical image that has the greatest score. Additionally, or alternatively, the platform 130 may determine a similarity score between a label of a medical image and a value of the structured data, and determine a match between the medical image and the structured data based on the similarity score. Additionally, or alternatively, the platform 130 may determine a medical image that depicts a region of interest having a greatest size, a greatest resolution, or the like. In this way, the medical image may be a medical image that permits a radiologist to readily and efficiently assess the findings in the radiology report because the medical image depicts the region of interest to which the finding corresponds.

As further shown in FIG. 4, the process 400 may include performing an action based on determining the medical image that depicts the region of interest of the subject (operation 470). For example, the platform 130 may perform an action based on determining the medical image that depicts the region of interest of the subject. According to an embodiment, the platform 130 may perform the action by displaying the radiology report and the determined medical image via a user interface of the user device 120. In this way, a radiologist may review the radiology report and the determined medical image that depicts the region of interest of the subject. Further, in this way, the radiologist might not need to review a large number of medical image datasets and corresponding medical images to identify the medical image that depicts the region of interest. Accordingly, the embodiments herein may reduce a number of queries to the medical image database, which thereby conserves device, database, and network resources.

According to another embodiment, the platform 130 may perform the action by generating a data entry that correlates the radiology report with the determined medical image. The data entry may be stored in the medical image database 140, the radiology report database 150, or the like. In this way, when a radiologist views the radiology report, the radiology report may already be correlated with the determined medical image, which permits the radiology report and the determined medical image to be displayed via a user interface of the user device 120. In this way, a radiologist may review the radiology report and the determined medical image that depicts the region of interest of the subject. Further, in this way, the radiologist might not need to review a large number of medical image datasets and corresponding medical images to identify the medical image that depicts the region of interest. Accordingly, the embodiments herein may reduce a number of queries to the medical image database, which thereby conserves device, database, and network resources.

According to another embodiment, the platform 130 may perform the action by transmitting an alert to a user device 120 based on the radiology report and the determined medical image. In this case, the alert may identify a diagnosis in the radiology report, a finding of a radiologist, or the like. As another example, the platform 130 may perform the action by automatically scheduling an appointment for a follow-up medical image test, for medical imaging, for a medical procedure, or the like. In this case, the platform 130 may identify a medical practitioner to perform the follow-up, to perform the medical imaging, to perform the medical procedure, or the like, and schedule the appointment accordingly. As another example, the platform 130 may perform the action by transmitting the radiology report and the determined medical image to a user device 120 associated with another medical personnel, to a user device 120 associated with the subject, or the like. As another example, the platform 130 may perform the action by transmitting the radiology report and the determined medical image to the medical image database 140, to the radiology report database 150, or the like.

Although FIG. 4 depicts particular operations and a particular sequence of operations, it should be understood that other embodiments may include different operations or differently arranged operations than as shown in FIG. 4.

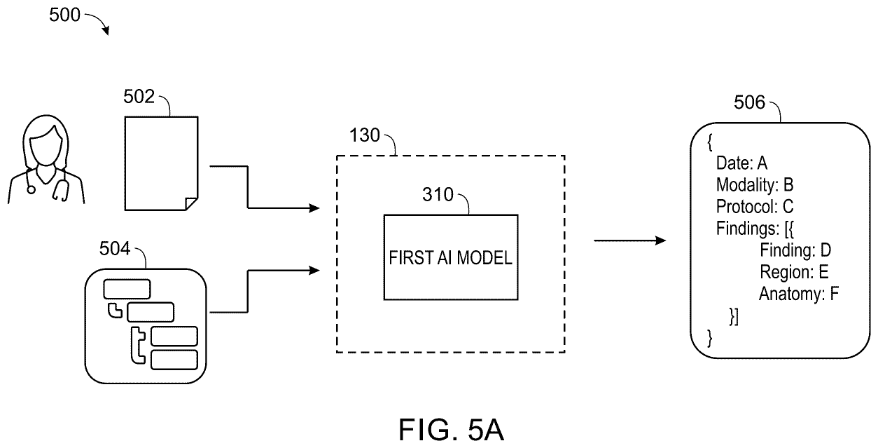

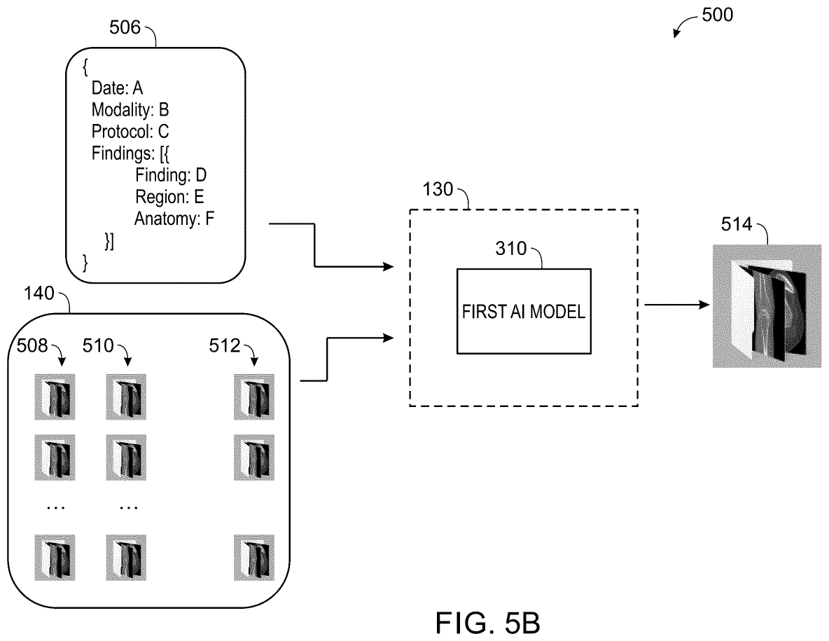

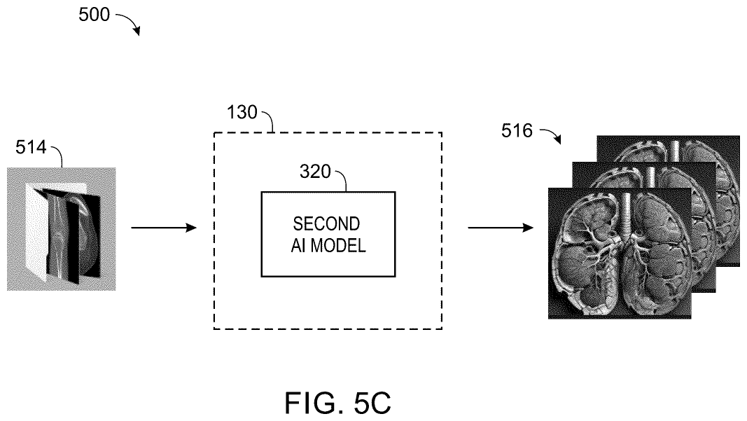

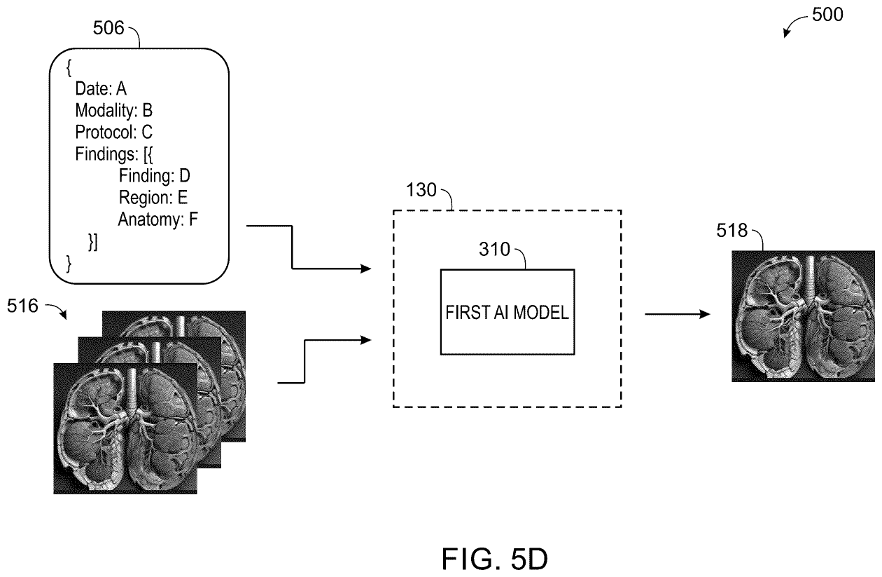

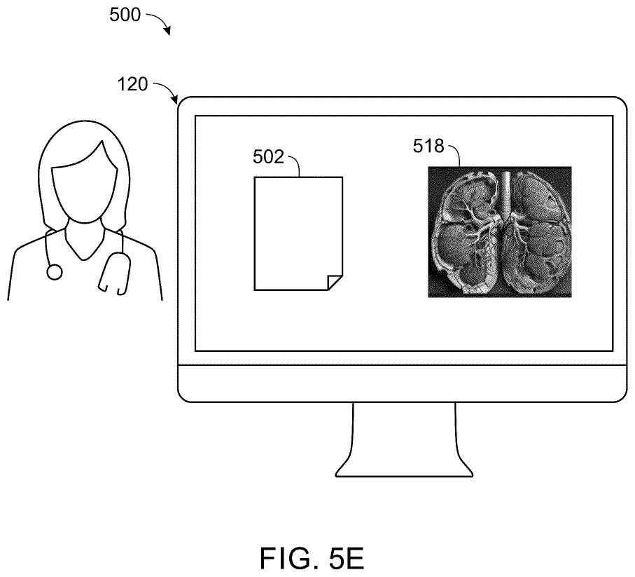

FIGS. 5A-5E are diagrams of an example process 500 for determining a medical image corresponding to a radiology report using AI models. As shown in FIG. 5A, the platform 130 may receive a radiology report 502 including a finding of a radiologist with respect to a region of interest of a subject. Further, as shown in FIG. 5A, the platform 130 may receive a configuration instruction 504 that identifies a predetermined set of fields of the radiology report for which to generate structured data. As further shown in FIG. 5A, the platform 130 may generate, using the first AI model 310, structured data 506 including a set of predetermined fields and a set of corresponding values extracted from the radiology report 502. For example, as shown, the structured data 506 may include a date field, a modality field, findings fields, etc., that include corresponding values extracted from the radiology report 502. As shown in FIG. 5B, the platform 130 may determine, using the first AI model 310, a medical image dataset 514 of the subject corresponding to the radiology report 502 from among a plurality of medical image datasets 508, 510, and 512 of the subject stored in a medical image database 140 using the structured data 506. For example, the platform 130 may determine that the medical image dataset 514 includes a same modality and a same date as included in the structured data 506. As shown in FIG. 5C, the platform 130 may segment, using the second AI model 320, a set of regions of each medical image of the medical image dataset 514 and label, using the second AI model 320, the set of regions of each medical image of the medical image dataset 514. The platform 130 may generate labelled and segmented medical images 516. As shown in FIG. 5D, the platform 130 may determine, using the first AI model 310, a medical image 518 of the medical image dataset 514 that depicts the region of interest of the subject using the structured data and the labelled and segmented set of regions included in the labelled and segmented medical images 516. As shown in FIG. 5E, the platform 130 may perform an action based on determining the medical image 518 that depicts the region of interest of the subject. For example, as shown, the platform 130 may display the radiology report 502 and the medical image 518 via a user interface of the user device 120.

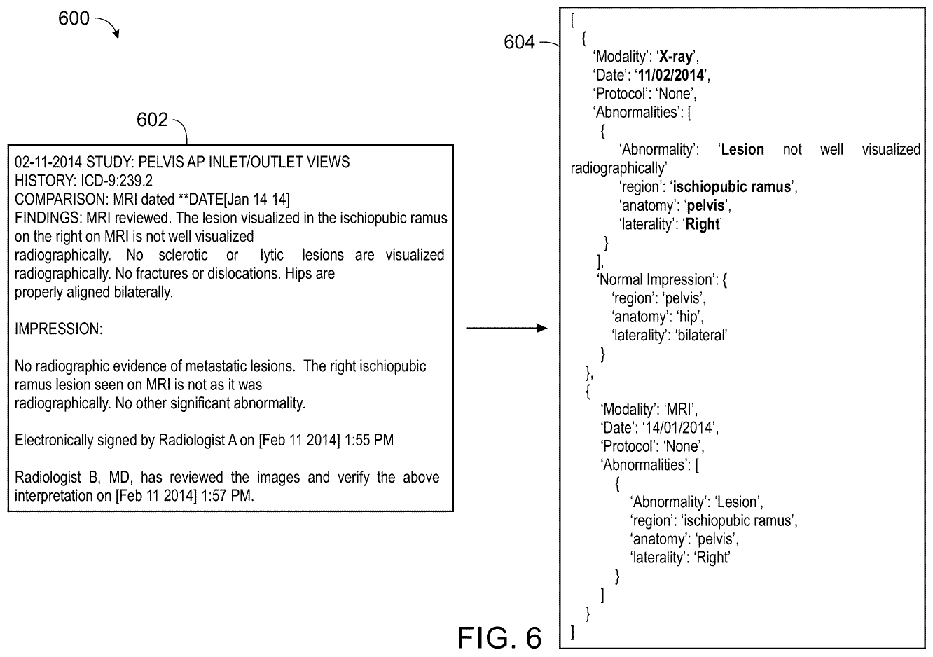

FIG. 6 is a diagram of an example process 600 for generating structured data including a set of predetermined fields and a set of corresponding values extracted from a radiology report. As shown in FIG. 6, a radiology report 602 may include one or more sections. For example, the radiology report 602 may include a type of medical imaging test section that identifies a type of imaging modality (e.g., X-ray) for the medical imaging test, a date of the medical imaging test (e.g., Feb. 11, 2014), a comparison section that includes information from a previous medical imaging test (e.g., an MRI), a findings section that identifies what the radiologist sees in the medical image (e.g., “MRI reviewed. The lesion visualized in the ischiopubic ramus on the right on MRI is not well visualized radiographically. No sclerotic or lytic lesions are visualized radiographically. No fractures or dislocations. Hips are properly aligned bilaterally”), and an impression section that identifies a summary of the findings (e.g., “No radiographic evidence of metastatic lesions. The right ischiopubic ramus lesion seen on MRI is not as it was radiographically. No other significant abnormality.”). As further shown in FIG. 6, the platform 130 may generate structured data 604 based on the radiology report 602. The structured data 604 may include a set of predetermined fields and corresponding values extracted from the radiology report 602. For example, the structured data 604 may include a modality field and a corresponding value (e.g., “X-ray”), a date field and a corresponding value (e.g., “Feb. 11, 2014”), a protocol field and a corresponding value (e.g., “none”), an abnormality field for a finding and a corresponding value (e.g., “lesion not well visualized radiographically”), a region field for the finding and a corresponding value (e.g., “ischiopubic ramus”), an anatomy field for the finding and a corresponding value (e.g., “pelvis”), a laterality field for the finding and a corresponding value (e.g., “right”), an impression field and a corresponding value (e.g., “normal”), a region field for the impression and a corresponding value (e.g., “pelvis”), an anatomy field for the impression and a corresponding value (e.g., “hip”), a laterality field for the impression and a corresponding value (e.g., “bilateral”), a modality field for a previous medical imaging test and a corresponding value (e.g., “MRI”), a date field and a corresponding value (e.g., “Jan. 14, 2014”), a protocol field and a corresponding value (e.g., “none”), an abnormality field for a finding and a corresponding value (e.g., “lesion”), a region field for the finding and a corresponding value (e.g., “ischiopubic ramus”), an anatomy field for the finding and a corresponding value (e.g., “pelvis”), a laterality field for the finding and a corresponding value (e.g., “right”).

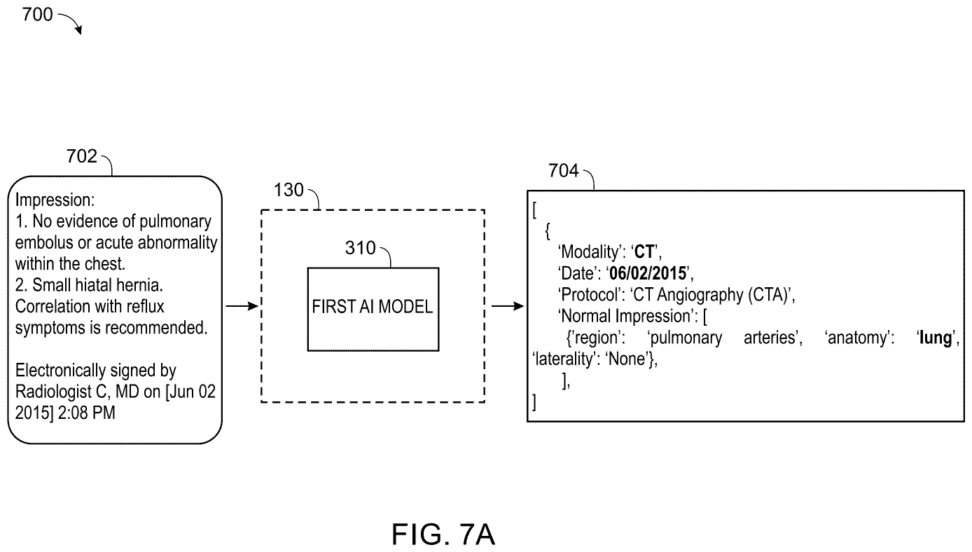

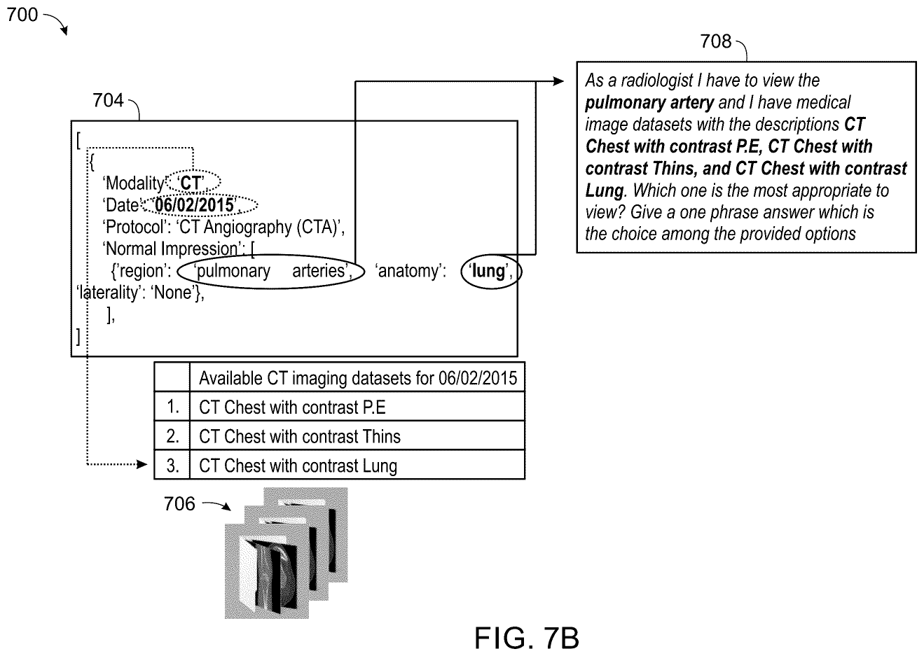

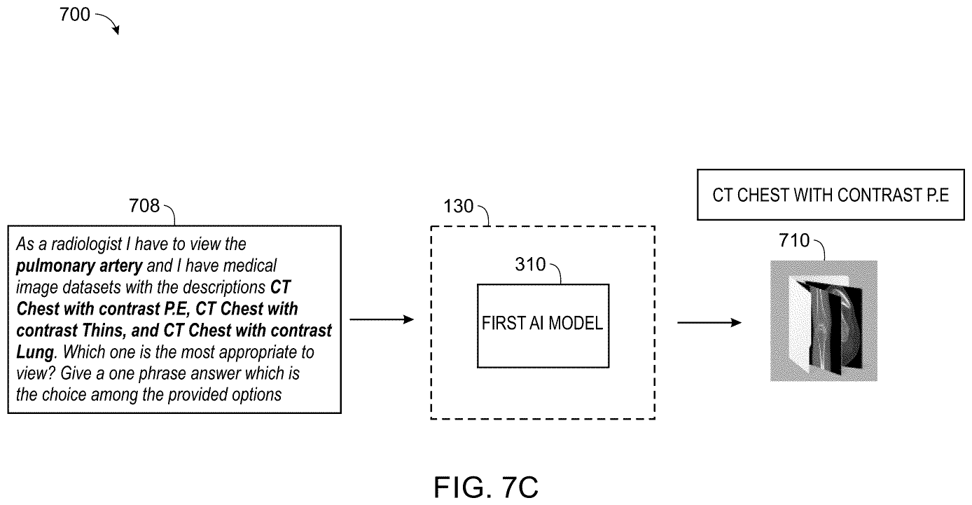

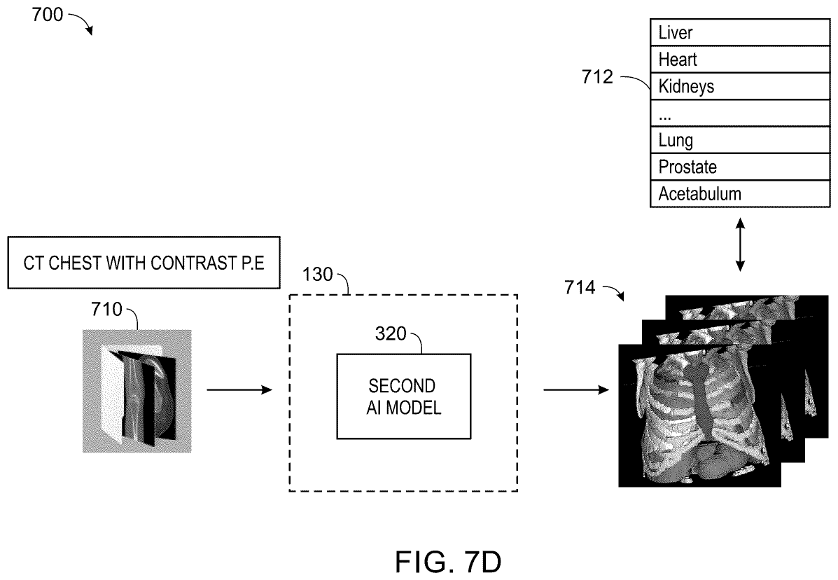

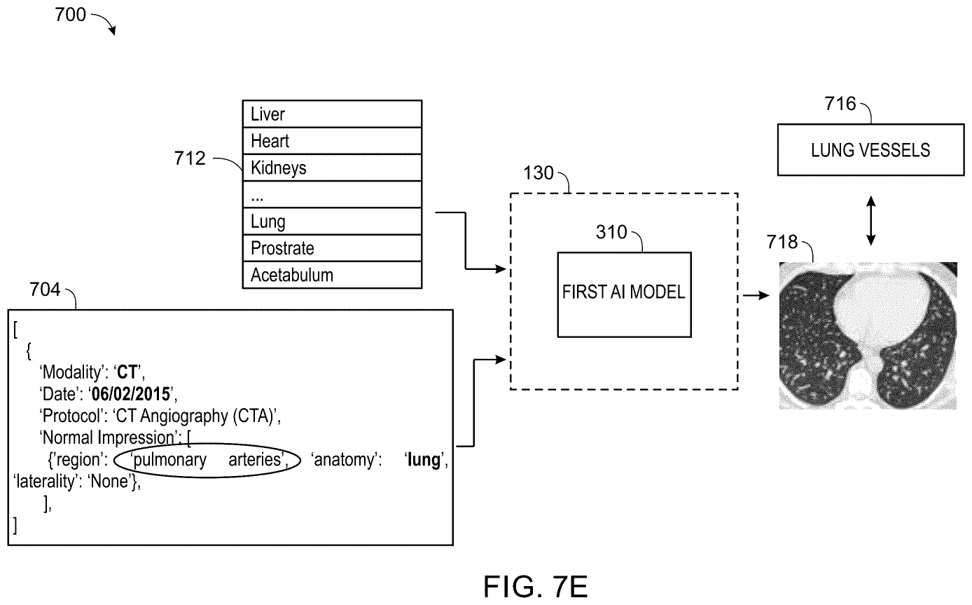

FIGS. 7A-7E are diagrams of an example process 700 for determining a medical image corresponding to a radiology report using AI models. As shown in FIG. 7A, the platform 130 may receive a radiology report 702 including a finding of a radiologist with respect to a region of interest of a subject (e.g., “1. No evidence of pulmonary embolus or acute abnormality within the chest. 2. Small hiatal hernia. Correlation with reflux symptoms is recommended.”). As further shown in FIG. 7A, the platform 130 may generate, using the first AI model 310, structured data 704 including a set of predetermined fields and a set of corresponding values extracted from the radiology report 702. For example, the structured data 704 may include an imaging modality field and a corresponding value (e.g., “CT”), a date field and a corresponding value (e.g., “Feb. 6, 2015”), a protocol field and a corresponding value (e.g., “CT Angiography (CTA)”), a finding field for a finding and a corresponding value (e.g., “normal impression”), a region field for the finding and a corresponding value (e.g., “pulmonary arteries”), an anatomy field for the finding and a corresponding value (e.g., “lung”), a laterality field for the finding and a corresponding value (e.g., “none”).

As shown in FIG. 7B, the platform 130 may determine, using the first AI model 310, a medical image dataset of the subject corresponding to the radiology report 702 from among a plurality of medical image datasets 706 of the subject stored in the medical image database 140 using the structured data 704. For example, the platform 130 may determine the medical image datasets 706 that include a date that matches a value of the date field of the structured data 704 and that include an imaging modality that matches a value of the imaging modality field of the structured data 704. Further, the platform 130 may generate a prompt 708 for the first AI model 130 that identifies the plurality of medical image datasets 706. For example, the prompt 708 may be “As a radiologist I have to view the pulmonary artery and I have medical image datasets with the descriptions CT Chest with contrast P.E, CT Chest with contrast Thins, and CT Chest with contrast Lung. Which one is the most appropriate to view? Give a one phrase answer which is the choice among the provided options.” As shown in FIG. 7C, the platform 130 may provide the prompt 708 to the first AI model 310. The first AI model 310 may output a selection of a particular medical image dataset 710 that satisfies the prompt 708.

As shown in FIG. 7D, the platform 130 may segment, using the second AI model 320, a set of regions of each medical image of the medical image dataset 710. Further, the platform 130 may label, using the second AI model 320, the set of regions of each medical image of the medical image dataset 710. For example, as shown, the platform 130 may input the medical image dataset 710 into the second AI model 320, and receive medical images 714 that include a labelled and segmented set of regions. The medical images 714 may be associated with labels 712. As shown in FIG. 7E, the platform 130 may determine, using the first AI model 310, a medical image 718 of the medical image dataset 710 that depicts the region of interest of the subject using the structured data 704 and the labelled and segmented set of regions. As shown, the first AI model 310 may match a value of a region field of the structured data 704 with a label 716, and determine a medical image 718 that is associated with the label 716.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A system comprising:
a memory configured to store instructions; and
one or more processors configured to execute the instructions to:
receive a radiology report including a finding of a radiologist with respect to a region of interest of a subject;
generate, using a first artificial intelligence (AI) model, structured data including a set of predetermined fields and a set of corresponding values extracted from the radiology report;
determine, using the first AI model, a medical image dataset of the subject corresponding to the radiology report from among a plurality of medical image datasets of the subject stored in a medical image database using the structured data;
segment, using a second AI model, a set of regions of each medical image of the medical image dataset;
label, using the second AI model, the set of regions of each medical image of the medical image dataset;
determine, using the first AI model, a medical image of the medical image dataset that depicts the region of interest of the subject using the structured data and the labelled and segmented set of regions; and
perform an action based on determining the medical image that depicts the region of interest of the subject.

2. The system of claim 1, wherein the performing the action comprises displaying the radiology report and the medical image that depicts the region of interest of the subject via a user interface of a user device.

3. The system of claim 1, wherein the performing the action comprises generating a data entry that correlates the radiology report with the medical image that depicts the region of interest of the subject, and storing the data entry in a database.

4. The system of claim 1, wherein the determining the medical image dataset of the subject comprises determining the medical image dataset based on a value of an imaging modality field included in the structured data.

5. The system of claim 1, wherein the determining the medical image dataset of the subject comprises determining the medical image dataset based on a value of a date field included in the structured data.

6. The system of claim 1, wherein the determining the medical image of the medical image dataset that depicts the region of interest comprises determining the medical image of the medical image dataset that depicts the region of interest based on a finding field included in the structured data and a label associated with the labelled and segmented set of regions.

7. The system of claim 1, wherein the determining the medical image dataset of the subject comprises determining the medical image dataset based on generating a prompt for the first AI model that identifies the plurality of medical image datasets.

8. A method comprising:
receiving a radiology report including a finding of a radiologist with respect to a region of interest of a subject;
generating, using a first artificial intelligence (AI) model, structured data including a set of predetermined fields and a set of corresponding values extracted from the radiology report;
determining, using the first AI model, a medical image dataset of the subject corresponding to the radiology report from among a plurality of medical image datasets of the subject stored in a medical image database using the structured data;
segmenting, using a second AI model, a set of regions of each medical image of the medical image dataset;
labelling, using the second AI model, the set of regions of each medical image of the medical image dataset;
determining, using the first AI model, a medical image of the medical image dataset that depicts the region of interest of the subject using the structured data and the labelled and segmented set of regions; and
performing an action based on determining the medical image that depicts the region of interest of the subject.

9. The method of claim 8, wherein the performing the action comprises displaying the radiology report and the medical image that depicts the region of interest of the subject via a user interface of a user device.

10. The method of claim 8, wherein the performing the action comprises generating a data entry that correlates the radiology report with the medical image that depicts the region of interest of the subject, and storing the data entry in a database.

11. The method of claim 8, wherein the determining the medical image dataset of the subject comprises determining the medical image dataset based on a value of an imaging modality field included in the structured data.

12. The method of claim 8, wherein the determining the medical image dataset of the subject comprises determining the medical image dataset based on a value of a date field included in the structured data.

13. The method of claim 8, wherein the determining the medical image of the medical image dataset that depicts the region of interest comprises determining the medical image of the medical image dataset that depicts the region of interest based on a finding field included in the structured data and a label associated with the labelled and segmented set of regions.

14. The method of claim 8, wherein the determining the medical image dataset of the subject comprises determining the medical image dataset based on generating a prompt for the first AI model that identifies the plurality of medical image datasets.

15. A non-transitory computer-readable medium storing instructions that, when executed by one or more processors, cause the one or more processors to:
receive a radiology report including a finding of a radiologist with respect to a region of interest of a subject;
generate, using a first artificial intelligence (AI) model, structured data including a set of predetermined fields and a set of corresponding values extracted from the radiology report;
determine, using the first AI model, a medical image dataset of the subject corresponding to the radiology report from among a plurality of medical image datasets of the subject stored in a medical image database using the structured data;
segment, using a second AI model, a set of regions of each medical image of the medical image dataset;
label, using the second AI model, the set of regions of each medical image of the medical image dataset;
determine, using the first AI model, a medical image of the medical image dataset that depicts the region of interest of the subject using the structured data and the labelled and segmented set of regions; and
perform an action based on determining the medical image that depicts the region of interest of the subject.

16. The non-transitory computer-readable medium of claim 15, wherein the performing the action comprises displaying the radiology report and the medical image that depicts the region of interest of the subject via a user interface of a user device.

17. The non-transitory computer-readable medium of claim 15, wherein the performing the action comprises generating a data entry that correlates the radiology report with the medical image that depicts the region of interest of the subject, and storing the data entry in a database.

18. The non-transitory computer-readable medium of claim 15, wherein the determining the medical image dataset of the subject comprises determining the medical image dataset based on a value of an imaging modality field included in the structured data.

19. The non-transitory computer-readable medium of claim 15, wherein the determining the medical image dataset of the subject comprises determining the medical image dataset based on a value of a date field included in the structured data.

20. The non-transitory computer-readable medium of claim 15, wherein the determining the medical image of the medical image dataset that depicts the region of interest comprises determining the medical image of the medical image dataset that depicts the region of interest based on a finding field included in the structured data and a label associated with the labelled and segmented set of regions.
